(19) United States
(12) Patent Application Publication
(10) Pub. No.: US 2025/0241530 A1
(43) Pub. Date: Jul. 31, 2025

(54) Self-Administered Adaptive Vision Screening Test Using Angular Indication

(71) Applicant: Northeastern University, Boston, MA (US)

(72) Inventors: Peter BEX, Concord, MA (US); Jan SKERSWETAT, Boston, MA (US)

(21) Appl. No.: 18/833,512

(22) PCT Filed: Feb. 13, 2023
(86) PCT No.: PCT/US2023/012959

§ 371 Date: Jul. 26, 2024

Related U.S. Application Data
(60) Provisional application No. 63/309,504, filed on Feb. 11, 2022.

(51) Int. Cl. A61B 3/032; A61B 3/00; A61B 3/02

(52) U.S. Cl. CPC A61B 3/032; A61B 3/0033; A61B 3/0041; A61B 3/022

(57) ABSTRACT

Self-administered tests for a broad range of visual functions use angular indications on a peripheral structure selected by the test subject to indicate the position of a visible feature of a stimulus. The tests are performed on a computer and use artificial intelligence to adapt and personalize the range of difficulty and type of test to the subject's vision issues. The tests can be used for diagnosis in the fields of optometry, ophthalmology, neurology, psychology, and psychiatry. The technology also can be used to determine a subject's optical prescription in combination with the use of ophthalmic lenses that are provided to the test subject.

### STATEMENT REGARDING FEDERALLY SPONSORED RESEARCH OR DEVELOPMENT

This invention was made with government support under Grant Number EY029713 awarded by the National Institutes of Health. The government has certain rights in the invention.

### BACKGROUND

In vision testing, many simple algorithms have been developed to change one stimulus property each trial. These algorithms generally decrease signal strength after the patient makes a correct response and increase signal strength after an incorrect response, slowly converging on an individualized criterion performance level (for review see Leek, 2001, Adaptive procedures in psychophysical research. Perception & Psychophysics, 63 (8), 1279-1292). These 1-dimensional algorithms are inefficient for measuring behaviors that vary along more than one dimension, as most complex behaviors do.

More recently, methods have been developed to select 2 or more stimulus properties each trial (e.g. Watson; QUEST+: A general multidimensional Bayesian adaptive psychometric method. Journal of Vision 2017; 17 (3): 10.), but these algorithms depend heavily on Bayesian assumptions that may be inaccurate for patient populations.

All the above methods aim to test a criterion level, typically 75% correct, which means that patients spend around 25% of the test making guesses about stimuli they cannot see. When stimuli vary along more than one dimension, the patient is not even sure what they are looking for.

There is a need for vision tests that can be administered quickly and in non-clinical settings, as well as tests that provide more informative data, can be adapted to the needs of the individual test subject, and changes over time and in response to therapeutic interventions.

### SUMMARY

The present technology provides comprehensive vision testing that is self-administered using a computer, tablet, or smart phone in a non-clinical or clinical setting and is based on an easy to understand and consistent set of charts (i.e., screen displays) using angular indications by the test subject using a pointer device or touch screen. The testing system uses artificial intelligence to adapt each test to the individual, thus increasing efficiency and resolution, resulting in personalized outcomes with higher diagnostic resolution in a short period of time (seconds to minutes for each test). No printed charts, booklets, or dedicated devices, spaces, or specially trained staff are required.

Current tests are deployed on designated devices, e.g. charts, booklets, screens with designated spaces, equipment hardware and training sessions for staff. The present technology vision tests e.g. for color, contrast, stereoacuity visual acuity are deployed on the same computer-based equipment, i.e. home computer, laptop, tablet, or mobile phone, and using the same general task. Hence, the present technology does not require additional stationary equipment, space, or training for staff for each test. The tests of the present technology use the same task, angular orientation indication, across all tests, and therefore do not require language familiarity or require patients to learn a different task for each test,

The present technology includes improved tests for visual acuity, low luminance acuity, color detection, color discrimination, stereoacuity (depth perception), interocular suppression, disability glare, perimetry, spatial-temporal contrast sensitivity, dark adaptation acuity and contrast sensitivity, and spatial contrast sensitivity as well as tests for visual functions for which there are currently no available tests, including center-surround suppression, motion perception, form perception, and supra-threshold contrast response. The tests can be used for diagnosis in the fields of optometry, ophthalmology, neurology, psychology, and psychiatry. The present technology also can be used to determine a patient's optical prescription (so-called “refraction”), in combination with the use of ophthalmic lenses that are provided to the test subject.

The present technology offers several advantages over traditional vision testing. For example, the software of the present technology can automatically select stimuli ranging from likely to unlikely to be seen, optionally for the specific test subject, to be presented in a vision test. The software also can use an artificial intelligence (AI) algorithm that automatically updates the stimuli used in a test based on the responses the patient makes to each stimulus selected by the algorithm. The type of stimuli also can be selected based on the visual function under investigation. For example, the algorithm can select stimulus sizes for a visual acuity test, colors for a color vision test, and other test stimuli related to the visual function being tested. For some visual outcomes, performance is known to depend on more than one stimulus property. For example, contrast sensitivity depends on both stimulus spatial frequency and contrast. In such cases, a sampling algorithm can select more than one property for the next stimulus, based on the patient's responses to previous stimuli. The algorithm can be implemented in any programming language and can operate on any desired operating system. It has been implemented, for example, in Matlab and run on a PC running Windows 10.

The present technology delivers standard outcomes in those visual function tests, for which there are comparable methods available, e.g., visual acuity threshold, but also delivers additional information about an observer's performance that are not captured by current tests.

Due to the large range of outcome data, the present technology also enables the use of machine learning methods to identify hidden pattern in longitudinal data sets for a single observer and for group comparison between two different groups. It allows the user to find clusters of tests that are predictive of vision function loss and that identify group differences.

An aspect of the present technology is a method for self-administered testing of one or more visual functions of a subject. The method includes: (a) providing a device having a graphical display and a user input; (b) displaying a series of cells on the display; (c) receiving subject responses through the user input; and (d) analyzing the subject responses to obtain a measure of said visual function for the subject. The cells each contain a visual stimulus, and typically only one visual stimulus, having a variable stimulus feature. Each cell contains the visual stimulus positioned within a perimeter feature, such as a circle, ellipse, square, rectangle, or other closed geometric feature having a displayed perimeter on which the subject can select an angular position. The visual stimulus possesses an angular indicator, such as a visible gap, structural alignment, or pointer at a unique angular position or orientation. The angular indicator varies from cell to cell in the series of cells in angular position with respect to the perimeter feature. Each different angular position is associated with a variation of the variable stimulus feature, wherein the variation is correlated with a visual function feature. The subject's responses indicate the subject's selection, for each cell, of a position on the perimeter feature of the cell corresponding to the angular indicator of the stimulus of the cell. For example, the subject can click with a mouse or other pointer device on the exact position on the perimeter feature, as perceived by the subject, that aligns with the angular feature of the visual stimulus.

The present technology can be further summarized with the following list of features.

1. A method for self-administered testing of one or more visual functions of a subject, comprising the steps of:
(a) providing a device having a graphical display and a user input;
(b) displaying a series of cells on the display; wherein the cells each comprise a visual stimulus having a variable stimulus feature; wherein each cell comprises the visual stimulus disposed within a perimeter feature; wherein the visual stimulus comprises an angular indicator that varies from cell to cell in angular position with respect to the perimeter feature, each different angular position associated with a variation of the variable stimulus feature correlated with a visual function feature;
(c) receiving subject responses through the user input, the responses indicating the subject's selection, for each cell, of a position on the perimeter feature of the cell corresponding to the angular indicator of the stimulus of the cell; and
(d) analyzing the subject responses to obtain a measure of said visual function for the subject.
   2. The method of feature 1, wherein the variable stimulus feature varies from cell to cell with respect to one or more of luminance, contrast, color, perceived depth, motion, flicker, spatial form, object recognition, center-surrounded stimulus, disability surrounded stimulus, object shape, object form, object size, stimulus feature position, stimulus feature angle, perceived interocular suppression, spatial resolution, spatial frequency, noise-defined depth, and sparse-pattern depth, presented either in the central or peripheral visual field.
   3. The method of any of the preceding features, wherein the variable stimulus feature varies within the series of cells over a range from difficult-to-detect to easy-to-detect for the subject.
   4. The method of any of the preceding features, wherein the series of cells is presented as one or more grids, each grid comprising two or more cells and sharing a common visual stimulus and variable stimulus feature, wherein the variable stimulus feature covers a range of values within each grid.
   5. The method of any of features 1-4, wherein the visual stimulus is a Landolt C, and the variable stimulus feature is selected from the group consisting of size, color, contrast to background, fill pattern and/or color, thickness, ratio of gap to thickness, and combinations thereof.
   6. The method of any of features 1-4, wherein the visual stimulus is an array or grating of structures comprising dots, bars, lines, or structures having other shapes, and wherein the variable stimulus feature is size, color, contrast to background, contrast to other structures in the array or grating, alignment of structures within the array or grating or a combination thereof.
   7. The method of feature 4, wherein artificial intelligence is used to adapt the visual stimulus, variable stimulus feature, or its range of variation to one or more previous responses of the subject, or information about the subject.
   8. The method of any of the preceding features, wherein a set of two or more grids are displayed sequentially, and each grid differs from others in the set by a type of visual stimulus or range of variation of the variable stimulus features.
   9. The method of feature 8, wherein a blank screen is displayed for a variable time interval between screens containing grids.
   10. The method of feature 9, wherein the blank screen is white and wherein the variable time interval is varied in the millisecond time domain, for intervals less than one second, and wherein the subject's flash adaptation is measured.
   11. The method of any of the preceding features, wherein the subject wears corrective lenses, anaglyph glasses, polarized glasses, a virtual reality headset, or uses a mirror system, two monitors, or any other dichotic-enabling system during testing.
   12. The method of any of the preceding features, wherein the method provides a measure of a visual function selected from the group consisting of visual acuity, visual acuity as a function of refractive errors, contrast sensitivity, glare sensitivity, motion perception, pattern perception, color detection, color discrimination, interocular suppression, monocular center, surround suppression, depth perception, form perception, supra-threshold contrast response, supra-threshold color response, equivalent noise thresholds
   13. The method of any of the preceding features, further comprising providing the subject with a plurality of corrective lenses having different refractive error correction and/or astigmatism correction, wherein the method is performed with the subject using one or more of the corrective lenses.
   14. The method of feature 13, wherein the opthalmic lenses or frames comprising the lenses are coded for recognition by the subject and/or by the device having a graphical display.
   15. The method of any of the preceding features, wherein the method is performed by an unassisted subject using a personal computer, laptop computer, tablet computer, or mobile phone in a non-clinical setting.
   16. The method of any of the preceding features, wherein results of the method are reported to a testing organization, ophthalmic optician, optometrist, ophthalmologist, psychologist, psychiatrist, neurologists, or medical doctor.
   17. The method of any of the preceding features, wherein a prescription for corrective glasses, contact lenses, or refractive surgery for the subject is produced.
   18. The method of any of the preceding features, wherein the test is optimized and personalized for the subject by performing two or more trials of the set of grids, wherein the range of the variable stimulus feature on the first trial are based on data from previous observers or on physical stimulus limits of the display, and wherein the range of the variable stimulus feature on subsequent trials is based on results from all previous grids for the subject.
   19. The method of any of the preceding features, wherein two are more variable stimulus features are varied simultaneously over the cells of a grid.
   20. The method of any of the preceding features, wherein the method is repeated after one or more time intervals.
   21. The method of any of the preceding features, wherein said analyzing comprises use of an angular error function.
   22. The method of any of the preceding features, wherein said analyzing comprises measuring the subject's orientation error bias as a function of target orientation.
   23. The method of any of the preceding features, wherein a plurality of visual functions are tested, and wherein said analyzing comprises performing cluster analysis.
   24. The method of any of the preceding features, wherein said analyzing comprises using a 2D or 3D mathematical function that describes the visual function to extract or predict an aspect of the subject's visual function from data supplied by the subject.
   25. The method of feature 24, wherein the 2D or 3D mathematical function describes contrast, spatial frequency, hue, or temporal stimulus change periods.
   26. The method of any of the preceding features, wherein the method is performed to aid in diagnosis of the presence, absence, or progression of one or more conditions selected from the group consisting of refractive error, keratoconus, amblyopia, age-related macular degeneration, glaucoma, diabetic retinopathy, color vision deficit, cataract, stroke, traumatic brain injury, brain lesion area V4, brain lesion area MT, brain lesion area FFA, Alzheimer's disease, Parkinson's disease, prosopagnosia, object agnosia, autism spectrum disorder, attention deficit disorder, neurometric response, psychotic disorders, post-traumatic stress disorder, obsessive compulsive disorder, Big 5 personality traits, albinism, prescription drug side effects, multiple sclerosis, visual snow syndrome, and retinitis pigmentosa.
   27. A device for performing the method of any of the previous features, the device comprising a graphic display, a user input, a processor, a memory, optionally wherein the processor and/or memory comprise instructions for performing said method.

### BRIEF DESCRIPTION OF DRAWINGS

FIG. 1A shows a schematic representation of an Angular Indication Measurement (AIM) test principle. The test subject reports the orientation of all C optotype gaps on a series of two grids. FIGS. 1B and 1C show embodiments of user interfaces for Visual Acuity (1B) and an example for Disability Glare using visual acuity as target stimulus (1C) tests. In these embodiments, visual acuity is measured as the smallest high contrast target whose orientation can be correctly identified. Stimuli are presented in a 4×4 cell grid, but other arrangements are possible. In each case, the observer indicates the apparent orientation of the target stimulus by clicking or touching the corresponding orientation on the outer response ring. The outer ring serves multiple purposes, it provides feedback concerning the reported orientation (the small, differently colored sector). The ring also serves to moderate disability, such as acuity in the presence of a glare source in FIG. 1C or under conditions of crowding (not shown). The observer can click the ring multiple times to adjust their report, in which case the feedback cue is updated. FIG. 1D shows how angular error is measured for observer responses requiring an angular indication.

FIGS. 2A-2H show illustrations of further stimulus implementations of the present technology. FIG. 2A shows random dot stimuli for a Motion, Pattern, and Equivalent Noise Perception test. FIG. 2B shows a Contrast Sensitivity test using a fixed size stimulus. FIG. 2C shows a test for Contrast Sensitivity as a function of Spatial Frequency (CSF) using bandpass filtered optotypes; FIG. 2D shows use of gratings for the same purpose. FIG. 2E shows a Color detection test. FIG. 2F shows a Interocular suppression test. FIG. 2G shows a Depth perception test FIG. 2H shows an example of a test using contrast-modulation stimuli.

FIGS. 3A and 3B show embodiments of display charts used in a Visual Acuity test. The subject indicates the perceived orientation of the centrally located letter “C” by clicking on the outer ring at the orientation of the gap in the “C”. The chart shown in FIG. 3A simulates ordinary light conditions (e.g., daylight), while the low luminance chart in FIG. 3B is used to measure visual acuity under low light conditions.

FIG. 4 shows application of angular error function to results from the Visual Acuity test shown in FIG. 3A. Circles show orientation report error as a function of letter size. For large letter sizes (logMAR>0.75, ETDRS equivalent 20/72), error is small and error increases as letter size decreases. When the observer cannot detect the stimuli, errors are random with a mean absolute error of 90° (π/2 radians) for stimuli with a 360° range and 45° (π/4 radians) for stimuli with a 180° range. The solid line shows the best fitting Angular Error Function (Equation 1), while the dashed lines show 95% confidence intervals at each stimulus level. Acuity equivalent values were immediately calculated and provided in logMAR and Snellen units. Additional information such as which eye was used, observer ID, noise, and the slope also were obtained using the same test results.

FIG. 5 shows an example of a defocus curve that can be used to determine peak visual acuity as corrected for a user. In the data shown, CSF acuity is plotted relative to best correction, as a function of refractive error added to best-correction for 8 observers. Visual acuity decreases with positive or negative defocus. The lines show linear regression fits, whose intersection shows optimal refraction.

FIGS. 6A-6C show a test and example outcomes for astigmatism. An eye with ideal or corrected-to-ideal optics perceives a radial pattern of different orientations equally (6A) and hence the window over the radial pattern should appear approximately circular. If the focus is asymmetric, then some orientations are better focused than others and the window may appear oriented to the right (6B) or left (6C). The orientation and shape of the ellipse can be used to estimate astigmatism and the cylinder correction necessary to correct it.

FIG. 7 shows an example of a chart for a Color Discrimination test. Two different colors are presented with various degrees of mixing using an equal-luminant cone contrast color space on a pedestal dynamic noise background. The user indicates the angular position of an axis best separating the colors.

FIG. 8A shows an example of a chart for a Contrast Sensitivity Function test. Alternating parallel bars of contrasting brightness (sinusoidal gratings) are shown with different levels of contrast and spatial frequency. The test subject indicates the angle best aligning with the bars. In FIG. 8B, the relationship between contrast and spatial frequency is depicted for a simulated test subject. The subject's peak contrast sensitivity and peak spatial frequency can be obtained from the data obtained using the chart of FIG. 8A and the relationship shown in FIG. 8B (see small arrows in FIG. 8B).

FIG. 9 shows an example of a chart for a Color Detection test. In this example the letter “C” is shown using a color of short wavelength using retinal cone-isolating, equal-luminance relative to the background on a pedestal dynamic noise background. The intensity of the color-contrast is varied, and the subject indicates the angular position of the gap.

FIGS. 10A and 10B show examples of charts for a Threshold versus Contrast test. In FIG. 10A the centrally located targets are achromatic, black-white sinusoidal gratings that are embedded in bandpass-filter noise background (pedestal contrast), made of light and dark spots of varying degrees of grayscale contrast, whereas in FIG. 10B the spots vary in contrasting colors. In both examples, the subject identifies the best angular dependence of an alignment pattern (e.g., grating or C optotype) against an increasingly noisy pedestal contrast background. FIG. 10C shows the theoretical relationship between target contrast and pedestal contrast, The black line indicates the dipper function. The minimum is a measure of internal noise and the slope is a measure of sensitivity. FIG. 10D shows the dipper function obtained from actual subject responses.

FIG. 11 shows an example of a chart for a Pattern and Motion Coherence and Equivalent Noise test. In this test, the stimulus can be either shown with all stimuli visible or with a hidden cell mode whereby cells become only visible if the observer's pointer hovers over a cell or uncovers a cell by tapping onto it to avoid that potentially peripherally seen stimuli are not visible as it is known that the peripheral visual field is more sensitive than for the central visual field. Hidden cells can be applied to any of the visual function tests described herein.

FIG. 12 shows an example of a chart for a Depth Perception test to interrogate binocular stereoacuity (i.e., 3D perception). In this example, two sets of differently colored dots (red and blue) are presented with varying horizontal disparities, i.e., different spatial locations of each dot. Anaglyph glasses separate left and right eyes stimuli, i.e., left eye sees red, right eye sees blue dots. Other image separating approaches may also be applied, e.g. mirror systems, polarized displays, two separate monitors, virtual reality headsets, and the like. The observer indicates the best angular position of a line dividing from an perceptually emerging three-dimensionally prominent bar that varies in orientation.

FIG. 13A shows an example of a series of charts for a Dark Adaptation test. In this test, a sequence of temporally varying stimulus and white charts presented to interogate contrast adaptation. Specifically, a chart for low contrast C optotypes is presented for a stimulus presentation duration, followed by a presentation of a white screen for an interstimulus duration, after which another stimulus presentation chart of the same duration is shown. This test simulates retinal dark adaptation, the period of which can be used to diagnose various visual conditions, such as age-related macular degeneration. In FIG. 13B, actual data of an observer is shown. Variation of the stimulus presentation duration depicted on the y-axis and the visual acuity thresholds for each duration on the x-axis are depicted and combine with lines. A two parameter regression function (not shown) can be fit to the data measuring the slope of the function as a measure of improvement over stimulus presentation duration.

FIG. 14 shows an example of a chart for a Surround Suppression test. Each cell of the grid contains a central target or stimulus which is surrounded by a bandpass-filtered noise area. In this example, the target is a sinusoidal grating that varies in orientation and contrast. The test subject identifies the orientation of the grating by indicating a position on the outer ring and the detection threshold is a measure of monocular suppression. Comparisons between stimuli with different degrees of surround stimuli are indicative of neural suppression that may be disrupted in neuro-ophthalmic conditions e.g., amblyopia.

FIG. 15 shows an example of a series of charts for a Perimetry test. Each cell contains a Landolt C that varies in orientation and contrast, plus a central C which is used as an indicator of peripheral gap orientation and as a fixation object. After the test subject evaluates the first chart, a blank screen is shown for an inter-stimulus duration, and then the initial stimulus is repeated. The tests measure peripheral sensitivity to the stimulus used, e.g. luminance ring as a function of spatial eccentricity and orientation. The stimuli are varied randomly in terms of stimulus intensity, e.g., orientation of the peripheral gap, and eccentricity relative to the fixation center.

FIGS. 16A and 16B show examples of a Range of Stimulus Detectability Improvement (ROSDI) for outcomes of two different observers' psychometric functions. The observer of FIG. 16A had a shallower slope and lower noise than the observer of FIG. 16B. It can be used for each AIM outcome and be applied to find ROSDI difference between groups or within an observer across time, e.g., pre- and post-treatment intervention.

FIGS. 17A-17D show four example plots for orientation indication error analysis. Each graph contains single data points (circles), the sinewave function (fitted solid line), the horizontal zero error line, and the midpoint of target orientation (vertical line) to aid visualization of phase. The y-axis shows the indicated error angle either positive or negative direction relative to the target orientation, or correct identification (along horizontal zero line). Relative proportion of error data [%] within each quadrant are also indicated. The x-axis shows the target orientations (0° to 359°). Outcome values of the model fit are shown on the top within each graph, namely biasori (slope), phase, amplitude, and goodness of fit (R²) of the function.

FIGS. 18A and 18B show results for agglomerative hierarchical cluster analysis. A battery of vision function tests, indicated on the horizontal axis, was performed on a group with albinism (18B) and age-matched typically sighted subjects (18A).

FIGS. 19A-19C show examples of prior art mathematical representations of visual functions that can be used to analyze data obtained with the present technology. FIG. 19A shows a 2D form of a spatial contrast sensitivity function. FIG. 19B shows a 3D form of the spatial contrast sensitivity function. FIG. 19C shows a color detection ellipse.

### DETAILED DESCRIPTION

The present technology provides a generalizable, self-administrable, and adaptive paradigm for scoring perceptual responses instantly and providing an estimate of visual sensitivity across the central and peripheral visual field.

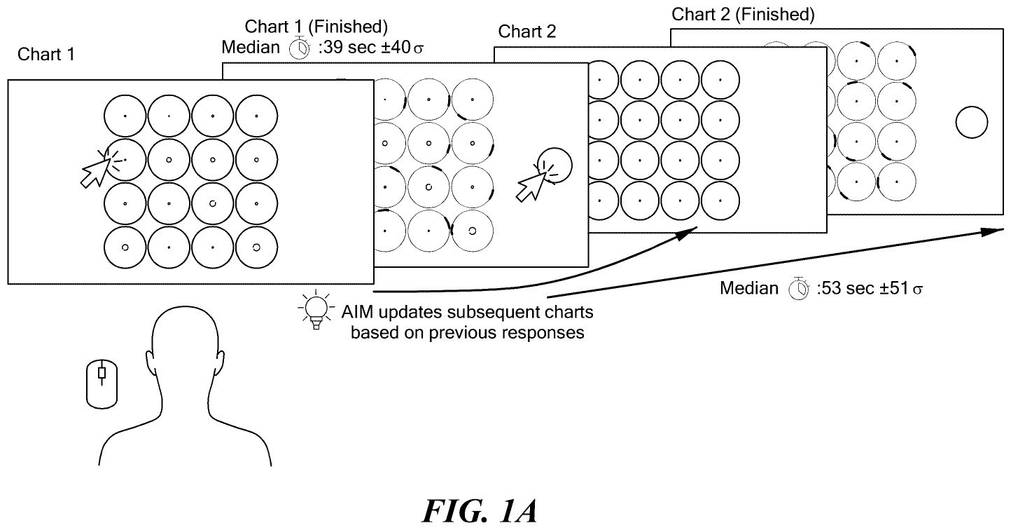

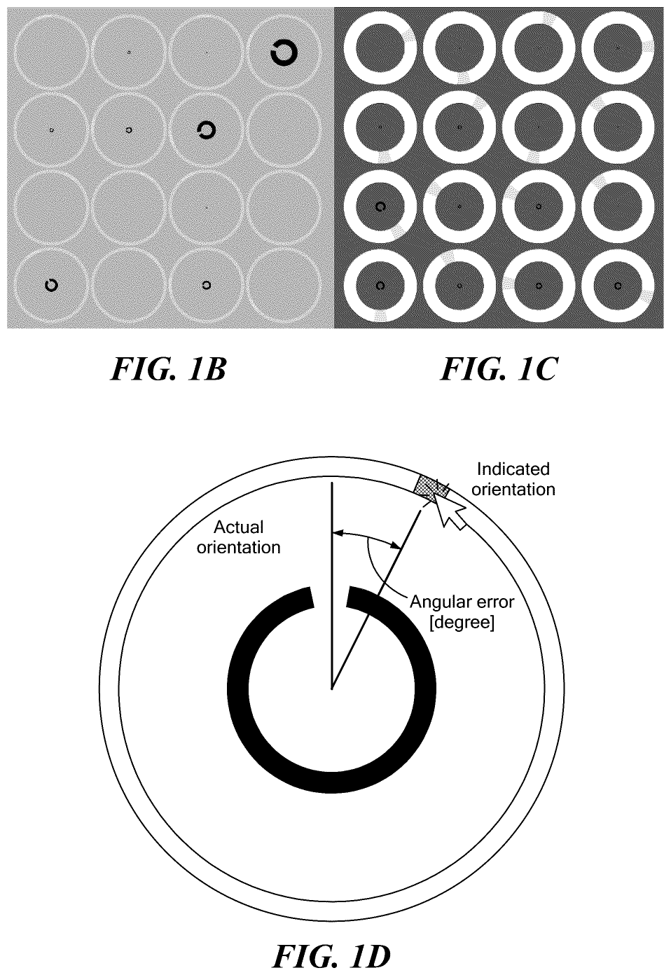

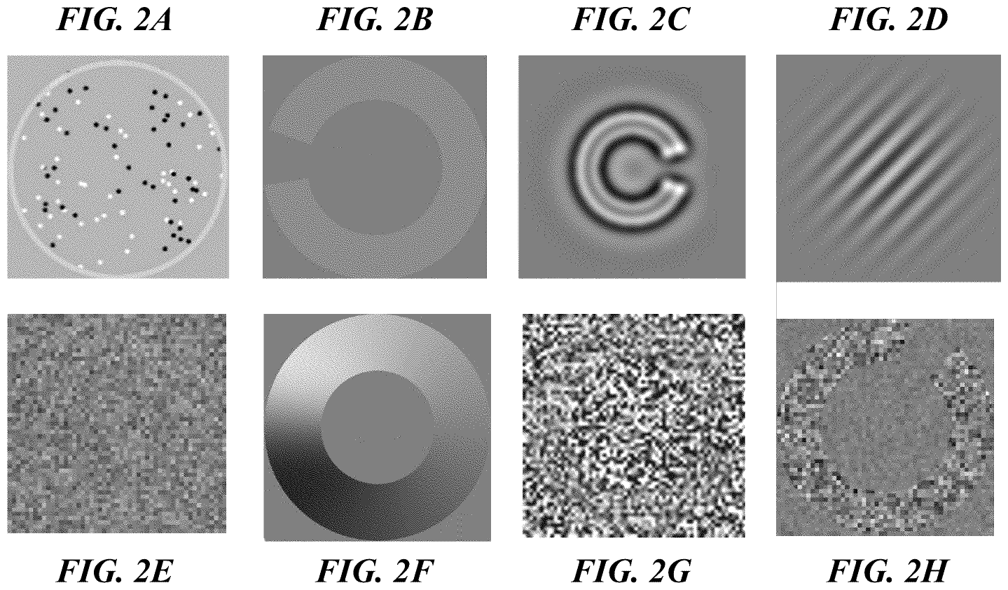

A sequence of stimuli is presented to the observer on a computer display. The stimuli may be presented in a matrix (as illustrated in FIGS. 1B and 1C) or singly (as illustrated in FIG. 2). The orientation or direction of movement of each stimulus can be either random or semi-random. The use of semi-random stimuli allows the testing of diagnostic orientations/directions, such as to quantify conditions such as astigmatism, visual distortions or metamorphopsia).

The present technology is assumption free and selects a range of stimuli that vary along one or more than one dimensions and selects stimuli that span a range from easy to difficult, so there is always a visible stimulus and so the patient knows what to search for. The intensity (e.g., size, contrast, depth, color saturation, signal:noise ratio etc.) of each stimulus is under the control of an algorithm that updates after each observer response. The stimuli can be defined by size (as illustrated in FIGS. 1B and 1C), luminance, contrast (FIG. 2B), spatial frequency (FIGS. 2C and 2F), color (FIG. 2E), depth, motion (FIG. 2A), spatial form. Binocular stimuli can be presented to test stereoacuity or interocular ratio (FIG. 2D). Stimuli that are in the form of a Landolt C or Landolt broken ring stimulus (i.e., presenting a structure shaped like the letter “C”) conform to vision testing standards in which the gap width is equal to the line width, both of which scale with the stimulus size. For visual field testing, the stimuli can be presented in the peripheral visual field, while the observer fixates on a central point. Compliant fixation may be enforced with an eye tracker or a central resolution task (e.g. an acuity stimulus that is only visible to the observer's foveal vision). Other visual properties that are diagnostic of different neuro-ophthalmic disorders can be instantiated in this paradigm.

Each stimulus can be surrounded by an outer response ring (see FIGS. 1B, 1C and 2A). The response ring provides feedback to the observer concerning their report and can also be used as a diagnostic moderator of stimulus visibility, e.g. as a glare source for cataract (FIG. 1C) or as crowding bars for amblyopia.

The human subject uses a computer mouse or a touch screen to indicate the perceived orientation or direction of movement of the stimulus by clicking or touching the corresponding orientation on the response ring. Feedback concerning the observer's report is provided on the response ring (e.g. by a change in color, luminance, line thickness etc., see FIGS. 1B, 1C). The participant can change their report by clicking or touching the response ring as often as they wish. After the observer has indicated the orientation of all stimuli on a screen, they click on a finish button next to the testing grid to continue with the next trial or finish the exam. Feedback concerning the accuracy of each report can be provided at this stage with visual or auditory display and may be gamified for engagement of children.

### Analysis Using Psychometric Error Function

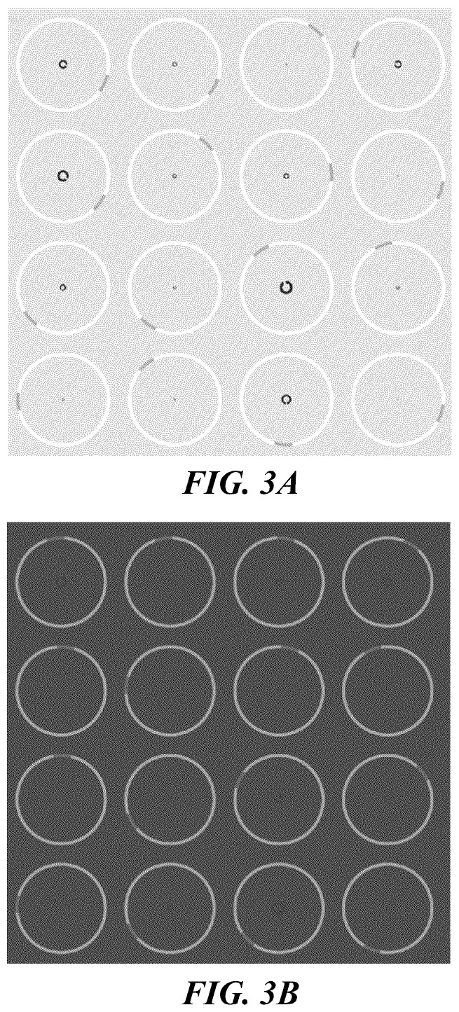

After the completion of each matrix, a computer algorithm calculates the angular error for each stimulus. Angular error is defined as the angular difference between the true and the reported orientation of each stimulus. A psychometric Angular Error Function is fit to the errors collected at all signal intensities, defined as:

$${τ}_{θ} ( s ) = ( {θ}_{i} + ( {θ}_{guess} - {θ}_{i} ) ) ( 0.5 + 0.5 * erf ( \frac{s - τ}{\sqrt{2} γ} ) ) \qquad ( 1 )$$

where θ_{guess} is the observer's orientation error for a guess response, which is fixed at π/2 for a stimulus with 360° range (e.g. C-type stimuli) or π/4 for a stimulus with 180° range (e.g. grating-type stimuli, FIG. 2D), s is signal intensity, T is a sensitivity threshold, e; is internal angular uncertainty, and γ is the slope of the function, see FIG. 3 for example.

Threshold performance for a given stimulus can be estimated from a criterion performance level on this function. Criterion performance levels include the value of T, which corresponds to the mid-point between best performance for a high intensity stimulus and guessing. Alternative criterion values can be specified to correspond to equivalent performance levels on standard visual function measures. For example, visual acuity with a Landolt C stimulus is defined as the line where at least ⅗ of the stimuli are identified correctly, and where the guessing rate is 25%. This point corresponds to an angular error of +34° clockwise and anticlockwise. Consider an example in which the true gap of a given Landolt C were orientated at 90° and an observer indicated the gap's location was at 115°. In this case, for calculating the Angular Error Function, the absolute response error angle is 25°.

In addition to threshold level, the parameter e; provides an estimate of internal angular uncertainty at best performance. This parameter is not measured with standard clinical tests (e.g. visual acuity, color, contrast, depth etc.), but may be informative about the patient's functional status. For example, patients may be able to identify the large letters of a visual acuity chart, but the images may be distorted or blurred by eye disease, but not badly enough to prevent identification of the letter or the orientation of a Landolt C or Tumbling E optotype. This loss of image quality would add angular error to the patient's orientation report that would not be detected with standard Visual Acuity charts.

Additionally, the slope parameter indicates the rate at which performance deteriorates with stimulus intensity. For most visual functions measured with standard clinical tests (e.g. visual acuity, color, contrast, depth), this parameter is not measured but may be informative about the patient's diagnosis.

Each observer's response can be rescored for equivalence with standard letter charts, such as ETDRS and Snellen Charts. For letter identification charts with 10 alternative SLOAN letters (C D H K N O R S V Z), the observer's 25° response error is equivalent to an Incorrect response, because each letter is equivalent to ±18° response range. However, for ETDRS charts with 4 alternatives (e.g. SLOAN letters H.O T V; Landolt C; or Tumbling E), the observer's 25° response error is equivalent to an Incorrect response, because each letter is equivalent to ±45° response range. This comparison illustrates the arbitrariness of discrete response scoring in standard charts, compared with continuous scoring in the present technology.

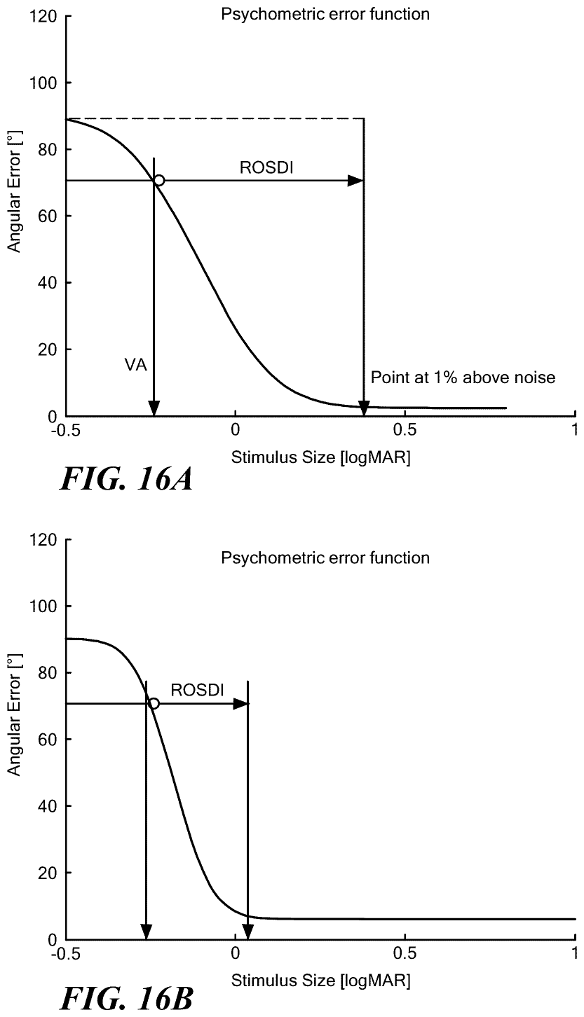

The slope of a psychometric function reveals the change of the detection probability across stimulus levels. It is an arbitrary value that relates the area of a function from where an observer cannot detect the signal (e.g., the signal is below the subject's visual acuity threshold) through where the observer always detects the signal (see FIGS. 16A and 16B). The present technology provides both thresholds (such as visual acuity threshold) and minimal indication error for each AIM outcome, which is a plateauing of suprathreshold performance. We calculated the distance from mean random response error (e.g. for C optotypes 90 degree) to an intensity value that corresponds to 1% above the observer's personal minimal indication error (FIGS. 16A, 16B). The following function applies for the angular error's corresponding intensity value n(x):

$$n ( x ) = τ \sqrt{γ} * ( \frac{erf ( C - 0.5 )}{- 0.5} ) \qquad ( 2 )$$

where τ is the sensitivity threshold (e.g., visual acuity threshold), γ is the slope of the function, and C the criterion point, here set at 0.01, i.e., 1% above minimum error angle (noise). The range between γ and n(x), here termed range of stimulus detectability improvement (ROSDI), was expressed in logMAR.

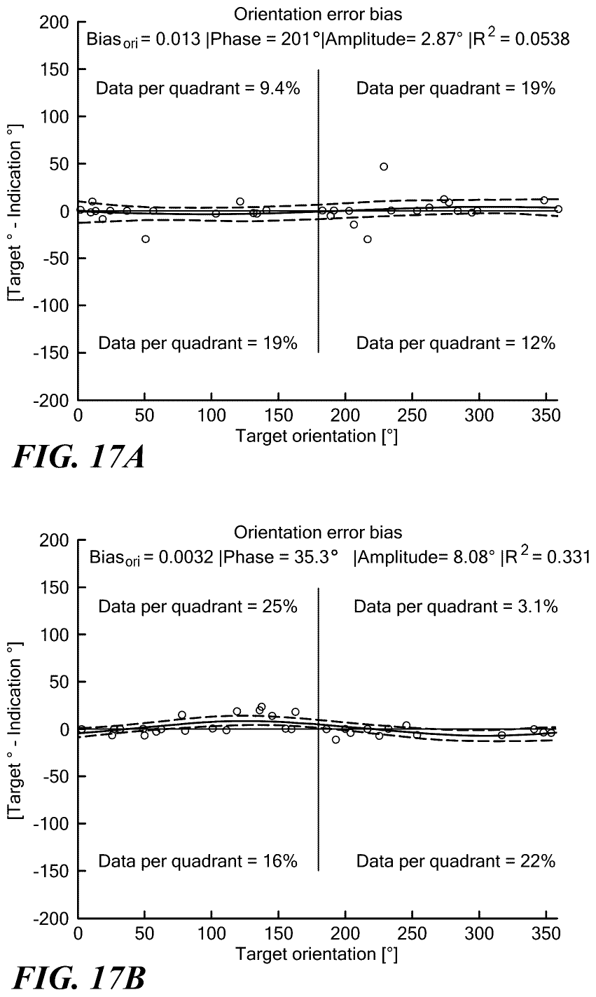

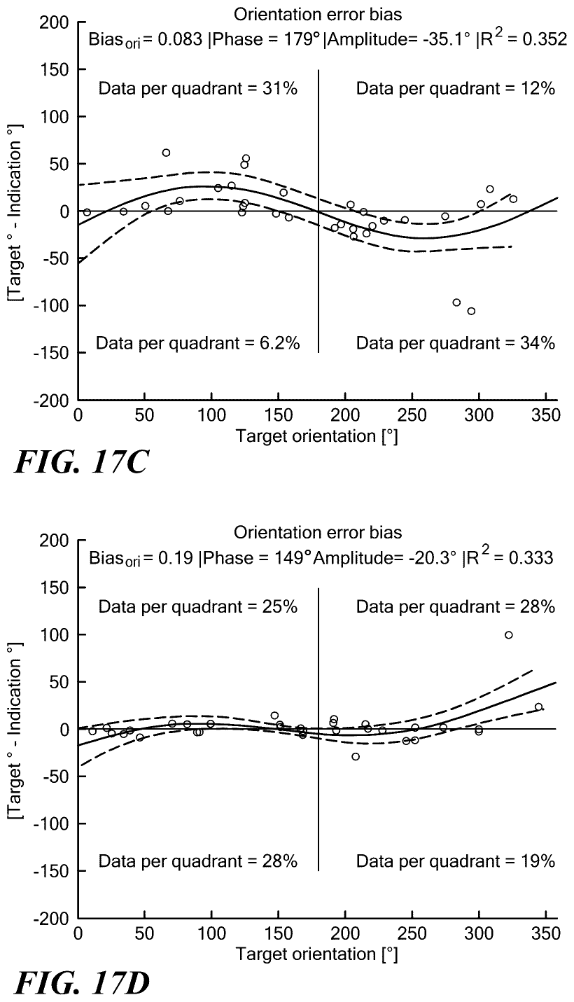

In an unbiased observer, orientation errors should be scattered randomly in either clockwise or counterclockwise orientation relative to the target orientation. However, if optical distortion is present due to astigmatism, a systematic bias should occur that follows a sinewave pattern when targets rotate 360°. If an observer is corrected for astigmatism, then distortions should be minimized unless neuro-ophthalmic distortion is present e.g., due to amblyopia(26). The present technology enables measurement of orientation indication bias (see FIGS. 17A-17D). Specifically, the orientation bias was calculated as a function of error direction (clockwise or counterclockwise relative to target orientation) and target orientation using a three free-parameter sinewave function to calculate phase, amplitude, and slope as shown below.

$$c = γ * ( θ guess \vert target - φ ) + sin ( θ guess \vert target - A ) \qquad ( 3 )$$

θ_{guess|target} is the observer's orientation error for a guess response and target gap orientation, Y is the slope (here referred to as orientation bias), o is the phase, and A is amplitude of the function. R² can be calculated to evaluate the goodness of fit.

FIG. 17A shows an example of data from an observer with relatively unbiased distribution of indication errors as indicated by the small amplitude and with unbiased distortion as indicated by Biasori resulting in a low goodness of fit R². FIG. 17B shows an observer with a mild orientation bias, indicated by both elevated amplitude and R². FIG. 17C shows an even stronger orientation bias indicated by the amplitude, as does FIG. 17D, but the latter's slope is more tilted. Anomalous slope can be indicative of a non-optical response distortion.

### Cluster Analysis

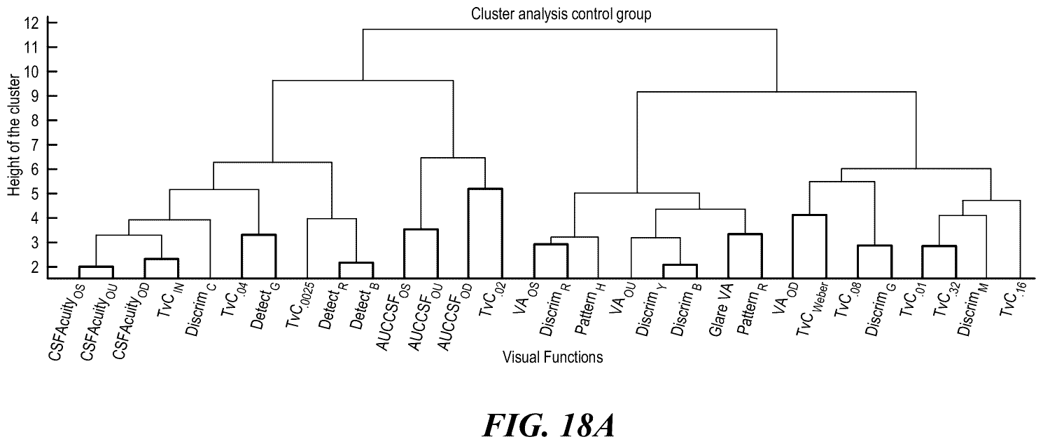

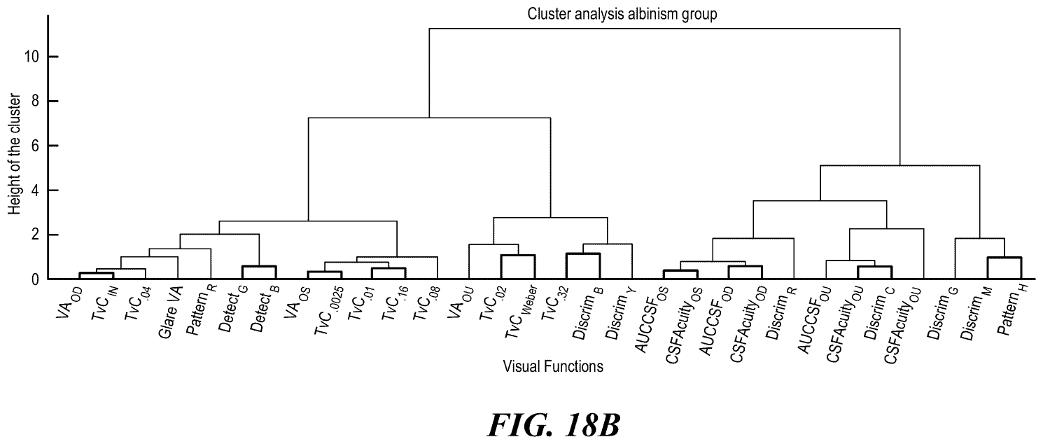

With the collection of larger data sets, new possibilities for data analysis emerge, specifically cluster analysis. In FIGS. 18A-18B, this analysis was deployed to identify clusters of output threshold values in two different groups of participants, a control group (FIG. 18A) and a group with albinism (FIG. 18B). This is merely an example of the type of cluster analysis that can be used for groups having a selected vision or other medical condition. Cluster analysis also can be employed for a single observer, for example when measuring a given battery of vision function tests over time to identify condition- and/or observer-specific trends in visual behavior.

### Application of Theoretical Representations of Visual Function

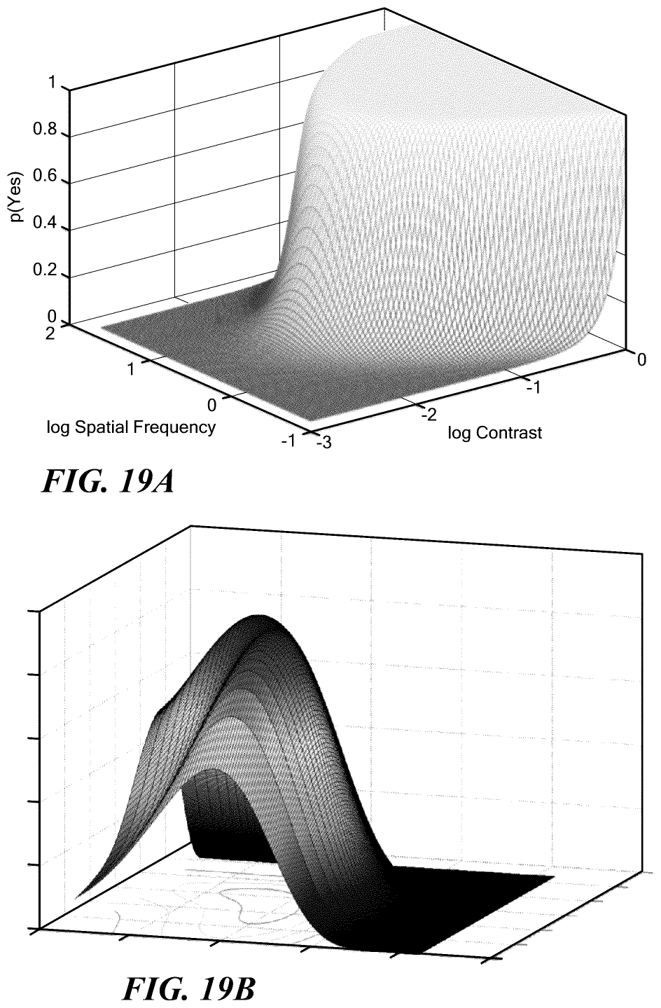

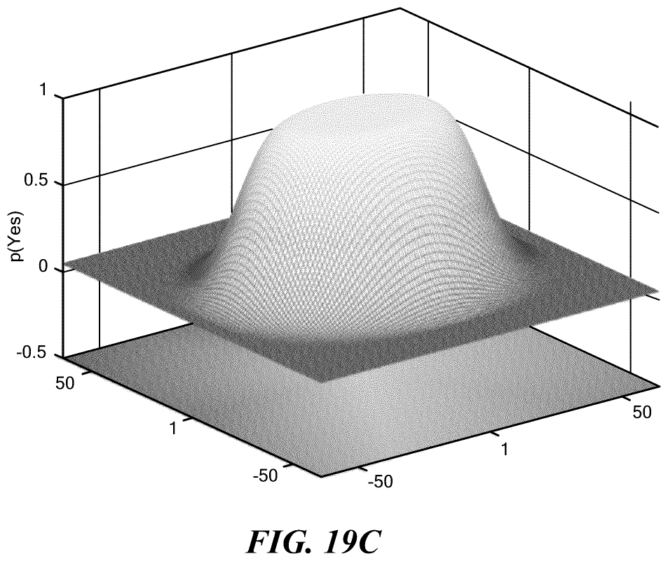

The technology described above provides many tests of a single visual function, such as spatial frequency for contrast and hue for color, that has a threshold whose value has important diagnostic value for a subject. In many cases, known theoretical relationships exist in form of a mathematical function which describes a threshold of interest for evaluating a subject's vision or neurological state. For example, the Spatial Contrast Sensitivity Function is shown in 2D form in FIG. 19A and in 3D form in FIG. 19B, and the Color Detection Ellipse is shown in FIG. 19C. These representations can be applied to the present technology in the context of machine learning, where they allow the estimation of a probability of a Yes response for any stimulus, and where they can be used to determine a threshold value. For example, spatial frequency and contrast stimuli can be modeled and predicted using the Spatial Contrast Sensitivity Function in 2D or 3D form, and hue and saturation stimuli can be modeled and predicted using the Color Detection Ellipse.

The use of these models can greatly accelerate the speed and efficiency of testing. In these cases, the range of stimuli on each chart can be specified by software based on the theoretical relationship and expected or actually input data for the subject, and can be used to cover the ongoing estimate of the potentially visible stimulus range (e.g., spatial frequency, hue or temporal frequency) as well as angular error. Summary data outputs from the three example cases include area under the log contrast sensitivity function, area under the threshold-versus contrast function, area under the chromatics sensitivity function, and volume under the spatio-temporal contrast sensitivity function. The three functions shown in FIGS. 19A-19C are just examples; other theoretical relationships, either known or developed for a specific visual function, can be applied. Machine learning can be used to refine the fit of collected data, or to refine the theoretical relationship, so as to improve performance of the testing system over time generally, or for a specific visual function, or for a specific test subject.

### Diagnostic Testing

The present technology provides a broad spectrum of individual tests that can be administered to a test subject either to generally assess the subject's visual function or in order to make a diagnosis of the presence, absence, or progression of a specific visual, neurological, or psychological condition. Such diagnoses can be used by medical professionals to recommend corrective lenses or specific medical treatments, or adjustments thereto.

Often, a specific combination of visual function tests can be used to diagnose a condition. The table below summarizes combinations of visual function tests according to the present technology that can be administered together to a subject, optionally at the same time, and the results used to diagnose the indicated condition.

| | | Visual Function Test | | | | | | |
|---|---|---|---|---|---|---|---|---|
| | | | Spatial | Temporal | | | | |
| | | Acuity | Contrast | Contrast | Color | Pattern | Motion | Depth |
| Condition | Refractive error ¹ | X | X | ✓ | ✓ | ✓ | ✓ | ✓ |
| Diagnosed | Keratoconus ² | X | X | ✓ | ✓ | ✓ | ✓ | ✓ |
| | Amblyopia ³ | X | X | ✓ | ✓ | X | X | X |
| | Age-related Macular | X | X | ✓ | ✓ | X | ✓ | X |
| | Degeneration ⁴ | | | | | | | |
| | Glaucoma ⁵ | ✓ | X | X | X | ✓ | X | ✓ |
| | Diabetic Retinopathy ⁶ | X | X | ✓ | ✓ | ✓ | ✓ | ✓ |
| | Color Vision Deficit⁷ | ✓ | ✓ | ✓ | X | ✓ | ✓ | ✓ |
| | Cataract ⁸ | ✓ | X | ✓ | X | ✓ | ✓ | ? |
| | Stroke* ⁹ | ✓ | X | X | X | X | X | X |
| | Traumatic Brain | ✓ | X | X | X | X | X | X |
| | Injury* ¹⁰ | | | | | | | |
| | Brain lesion area V4 ¹¹ | ✓ | ✓ | ✓ | X | X | ✓ | ✓ |
| | Brain lesion area MT ¹² | ✓ | ✓ | ✓ | ✓ | ✓ | X | ✓ |
| | Brain lesion area FFA ¹³ | ✓ | ✓ | ✓ | ✓ | ✓ | ✓ | ✓ |
| | Alzheimer's Disease ¹⁴ | X | X | X | X | ✓ | X | ✓ |
| | Parkinson's Disease ¹⁵ | X | X | X | X | ✓ | X | X |
| | Prosopagnosia ¹⁶ | ✓ | ✓ | ✓ | ✓ | ✓ | ✓ | ✓ |
| | Object Agnosia ¹⁷ | ✓ | ✓ | ✓ | ✓ | ✓ | ✓ | ✓ |
| | Autism Spectrum | ✓ | ✓ | ✓ | ✓ | X | X | ✓ |
| | Disorder ¹⁸ | | | | | | | |
| | Attention Deficit | ✓ | X | ✓ | X | ? | ? | ? |
| | Disorder ¹⁹ | | | | | | | |
| | Neurometric | ✓ | X | ✓ | X | ? | ? | ? |
| | Response ²⁰ | | | | | | | |
| | Psychotic disorders²¹ | ✓ | ✓ | ✓ | X | ? | X | ? |
| | Post-traumatic stress | ✓ | X | ✓ | ✓ | ? | ✓ | X |
| | disorder ²² | | | | | | | |
| | Obsessive compulsive | ? | ? | ? | ? | ? | X | ? |
| | disorder ²³ | | | | | | | |
| | Big 5 Personality | ✓ | ✓ | X | ✓ | ✓ | ✓ | ✓ |
| | traits ²⁴ | | | | | | | |
| | Albinism ²⁵ | X | X | X | X | ? | ? | ? |
| | Prescription drug | X | X | X | X | ? | ? | ? |
| | side-effects ²⁶ | | | | | | | |
| | Multiple Sclerosis²⁷ | X | X | X | X | ? | ? | X |
| | Visual snow | ✓ | ? | ? | ? | ? | ? | ? |
| | syndrome²⁸ | | | | | | | |
| | Retinitis pigmentosa²⁹ | ✓ | ✓ | ✓ | ✓ | ✓ | ? | ? |

| | | Visual Function Test | | | | |
|---|---|---|---|---|---|---|
| | | | | | Dark | Peripheral |
| | | Dipper | Glare | Suppression | adaptation | vision |
| Condition | Refractive error ¹ | ✓ | X | X | ✓ | X |
| Diagnosed | Keratoconus ² | ✓ | X | X | ✓ | X |
| | Amblyopia ³ | ✓ | ✓ | X | X | ✓ |
| | Age-related Macular | ✓ | X | X | X | ✓ |
| | Degeneration ⁴ | | | | | |
| | Glaucoma ⁵ | ✓ | X | X | X | X |
| | Diabetic Retinopathy ⁶ | ✓ | ✓ | X | ? | ✓ |
| | Color Vision Deficit⁷ | ✓ | ✓ | ✓ | X | ✓ |
| | Cataract ⁸ | X | X | X | ✓ | ✓ |
| | Stroke* ⁹ | X | X | ? | ? | X |
| | Traumatic Brain | X | X | X | X | X |
| | Injury* ¹⁰ | | | | | |
| | Brain lesion area V4 ¹¹ | ? | ? | ? | ? | ? |
| | Brain lesion area MT ¹² | ? | ? | ? | ? | ? |
| | Brain lesion area FFA ¹³ | ? | ? | ? | ? | ? |
| | Alzheimer's Disease ¹⁴ | ? | ? | ? | ? | ? |
| | Parkinson's Disease ¹⁵ | ? | ? | ? | ? | ? |
| | Prosopagnosia ¹⁶ | ? | ? | ✓ | ? | ? |
| | Object Agnosia ¹⁷ | ? | ? | ✓ | ? | ? |
| | Autism Spectrum | ? | ✓ | X | ? | ? |
| | Disorder ¹⁸ | | | | | |
| | Attention Deficit | ? | ? | X | ? | ? |
| | Disorder ¹⁹ | | | | | |
| | Neurometric | ? | ? | ? | ? | ? |
| | Response ²⁰ | | | | | |
| | Psychotic disorders²¹ | ? | ? | ? | ? | ? |
| | Post-traumatic stress | ? | ? | ? | ? | ? |
| | disorder ²² | | | | | |
| | Obsessive compulsive | ? | ? | ? | ? | ? |
| | disorder ²³ | | | | | |
| | Big 5 Personality | ✓ | ? | ? | ? | ? |
| | traits ²⁴ | | | | | |
| | Albinism ²⁵ | ? | X | X | ? | ✓ |
| | Prescription drug | ? | X | ? | ? | ✓ |
| | side-effects ²⁶ | | | | | |
| | Multiple Sclerosis²⁷ | X | ? | X | X | ✓ |
| | Visual snow | ? | X | ? | X | ✓ |
| | syndrome²⁸ | | | | | |
| | Retinitis pigmentosa²⁹ | ? | ✓ | ✓ | X | X |

### Example 1: Disability Glare

Owing to its speed, the present technology can be extended to define new quantitative metrics that are not practically possible with existing approaches. For example, Disability glare is a consequence of straylight from intraocular light scatter by optical imperfections (such as cataract, corneal disease, multifocal lenses, refractive surgery) and retinal diseases (such as glaucoma and age-related macular degeneration). Disability glare causes a subjective experience of discomfort and an objective perceptual deficit that are currently measured with cumbersome systems (light sources attached to standard letter acuity charts), slow matching paradigms (e.g. luminance matching) or expensive optical devices (e.g. C-Quant Stray Light Detector, $20,000). Of particular relevance to the present technology, when glare sources are attached to visual acuity charts, the intensity of the light source intensity is fixed and the distance from each optotype is not constant. These problems confound glare measurement. In the present technology, Acuity Glare (FIG. 1C) addresses both of these concerns, since the light intensity and distance from the test stimulus can be varied independently. This enables the development of new standards to meet an unmet need for standardization in Glare testing.

### Example 2: Refraction

At least 2.3 billion people have poor vision due to refractive error¹. Refractive error is especially prevalent in southeast Asia where 98% of young adults have myopia². Refractive error correction is the primary reason for vision screening and traditionally requires a visit to an optometrist. Recent business models have disrupted the $140B eyewear industry with direct to customer sales (e.g., Warby Parker)³. However, this direct model still requires the customer to obtain a prescription from an optometrist, which minimizes the potential market impact of this transformative approach. With the present technology, Refraction allows users to complete a self-administered refraction test in their own home without the need to attend a clinic. This approach addresses the market opportunity separating eyewear customers and online spectacle vendors.

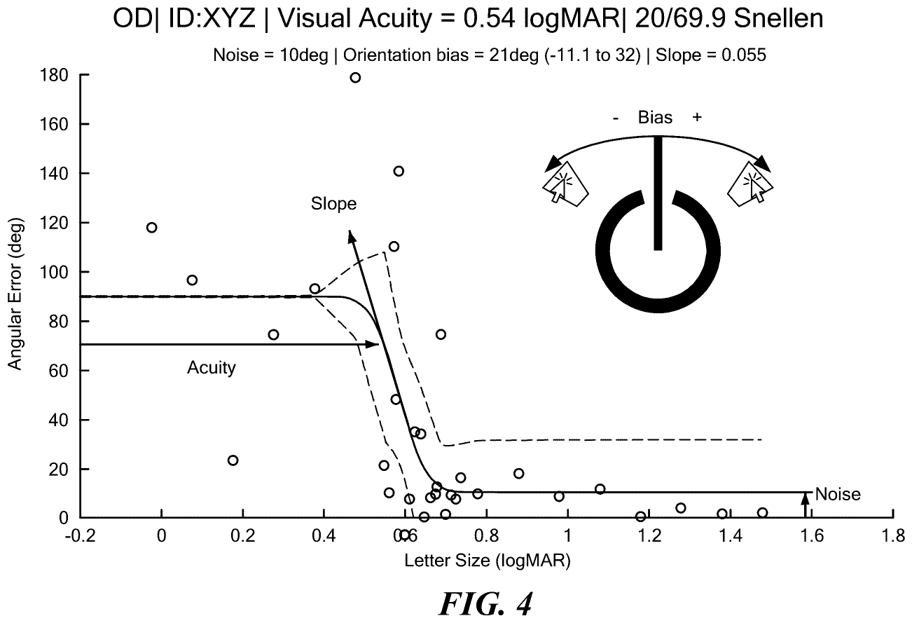

In the present technology, Refraction utilizes the Visual Acuity test illustrated in FIG. 1A. A set of spectacles with different refractive corrections (e.g. 21 pairs from −5 to +5 diopters) can be sent to the home of users, each differentiated, for example, by a unique color, number and barcode on the front of the spectacle's frame. The barcodes allow specific spectacles to be automatically identified by a webcam and image processing. The barcodes can also be used to estimate the user's viewing distance from the computer screen. The method can include the following steps:
1) An algorithm can select a refraction for the next visual acuity test. The first test refraction can be based on the user's existing prescription, if known; on typical data for the user's demographics if known; or on the population average (−1 diopters) if nothing is known about the user. The refraction on subsequent tests can be based on Visual Acuity estimates from the refractive correction tested on previous tests. Until the user's Visual Acuity is logMAR 0 (equivalent to 6/6 or 20/20), then refractive correction is necessary. Linear regression fits to Visual Acuity estimates indicate how each change of refractive correction affects visual acuity. If visual acuity decreases following a change in refractive correction, then the refractive correction on the next trial will be in the opposite direction.
2) The user can be instructed to wear the spectacles chosen in step 1 (verified by webcam-based image processing on the barcode) and perform a Visual Acuity test (which takes about 30 seconds to complete).
3) An algorithm can estimate the user's visual acuity with the current refraction. Example Visual Acuity estimates for 8 observers and 6 refractive errors are shown in FIG. 4.
4) Steps 1-3 can be repeated until an apex with at least two estimates above and below the peak visual acuity are estimated (see FIG. 4).
5) The intersection of the bilinear regression fits indicates best refraction for the user. The intersection is reported to the user and used for the prescription. Note that if visual acuity is worse than logMAR 0 with any refractive correction, this could indicate a serious visual problem that would be referred to an optometrist or ophthalmologist.

### Example 3: Astigmatism

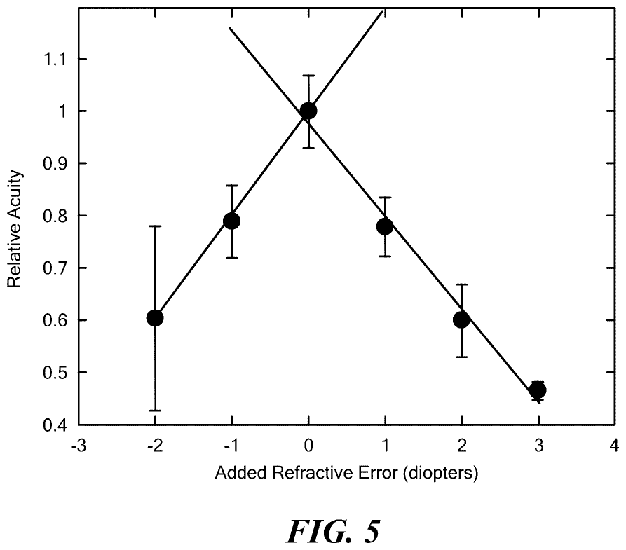

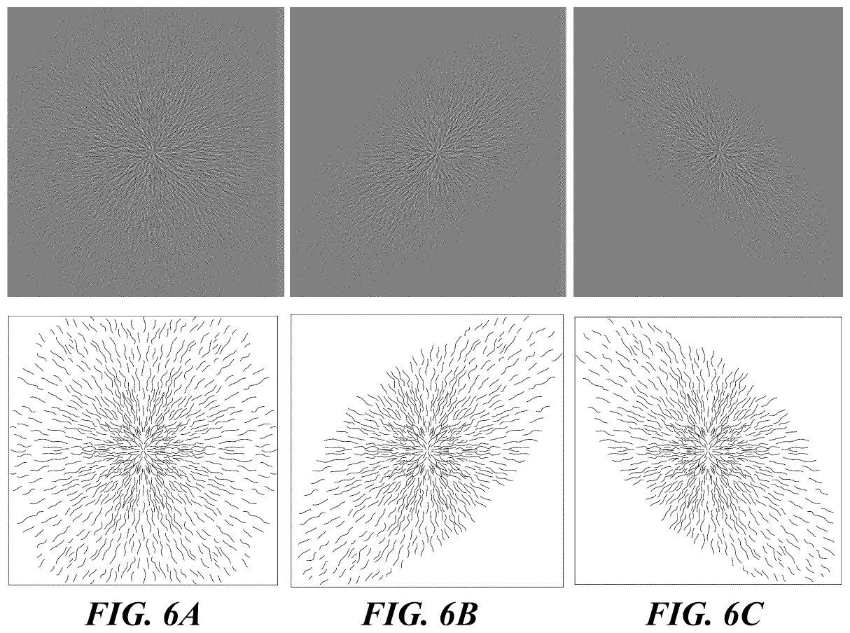

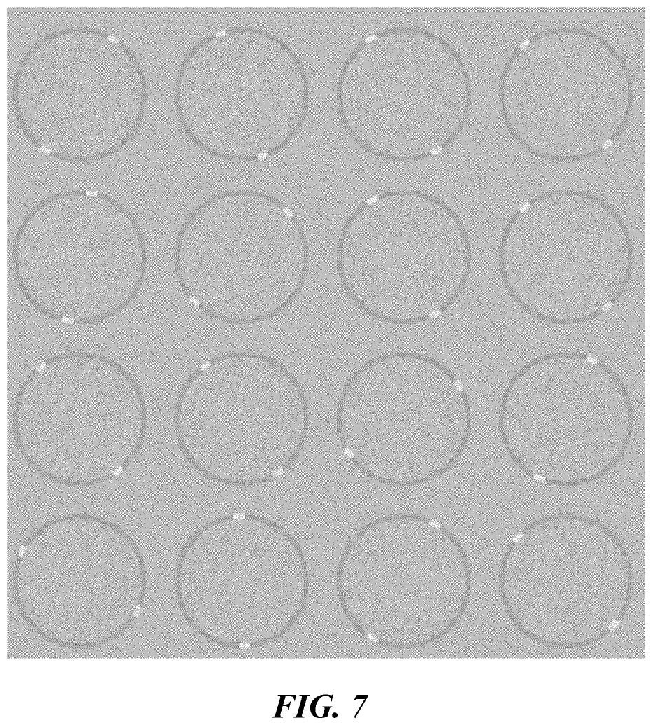

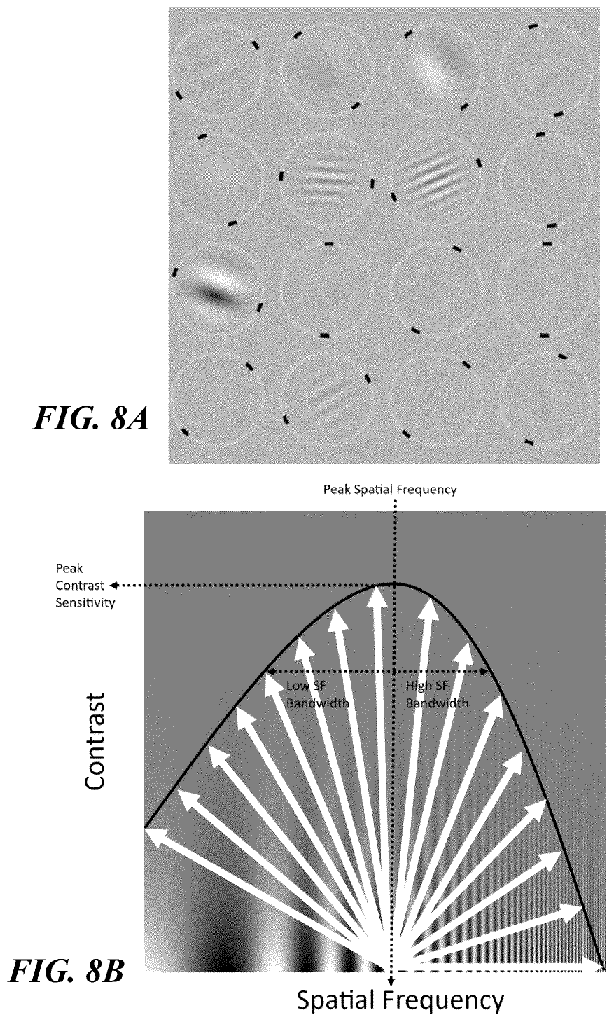

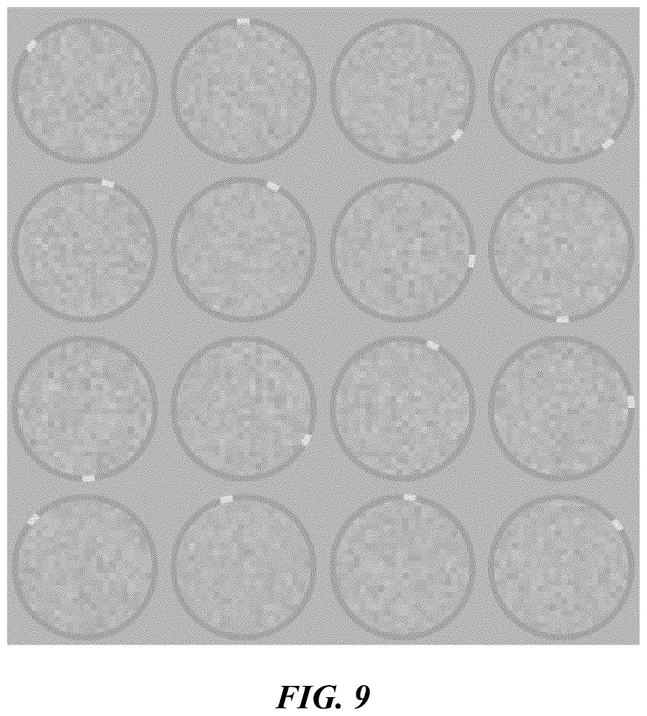

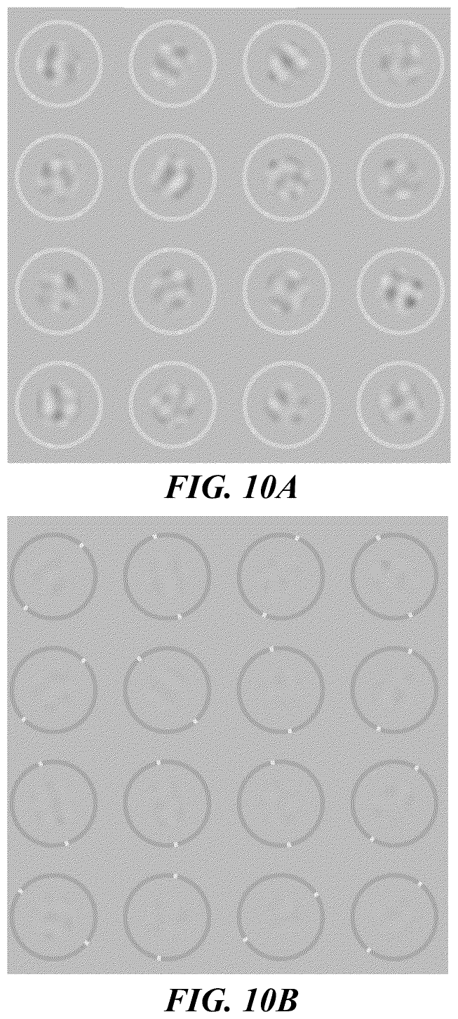

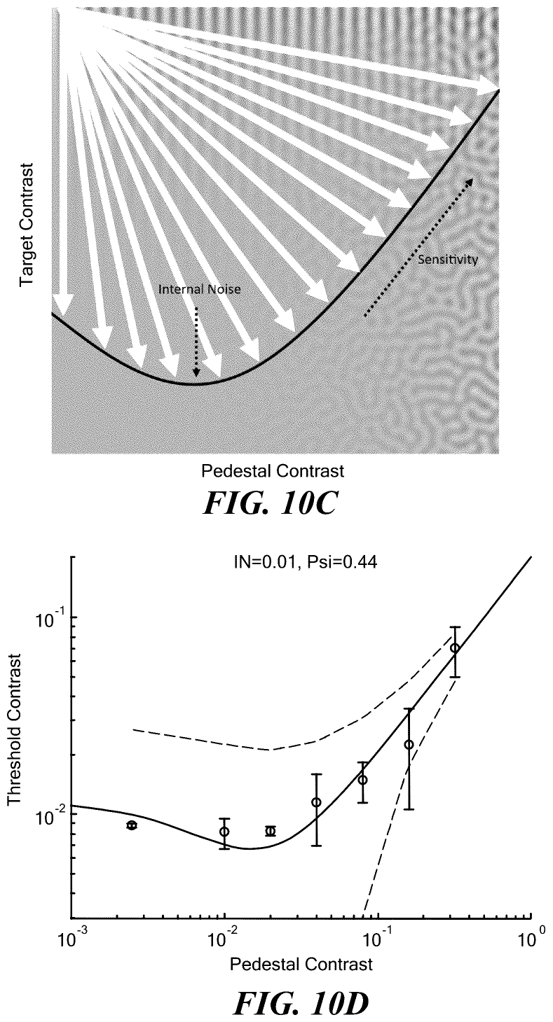

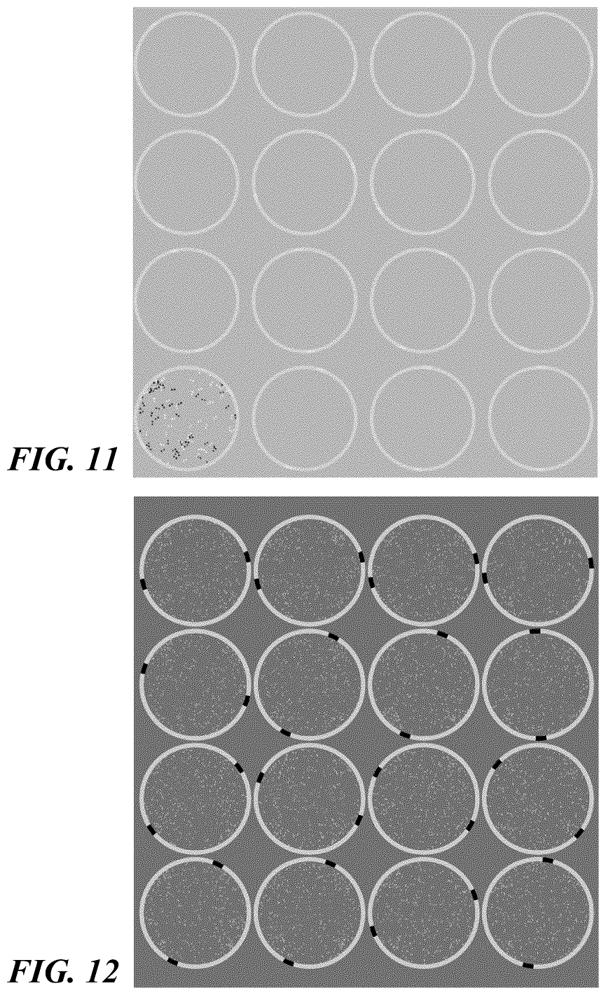

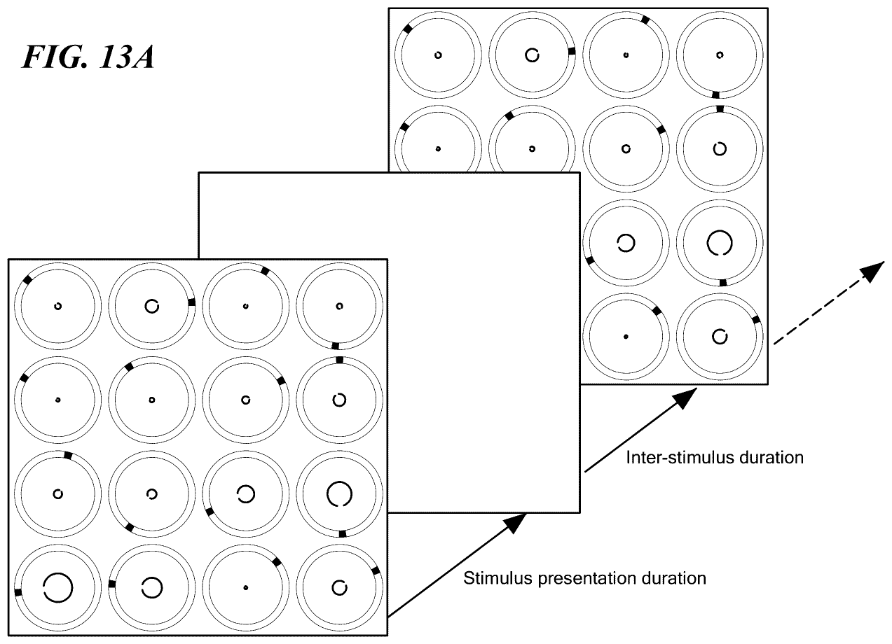

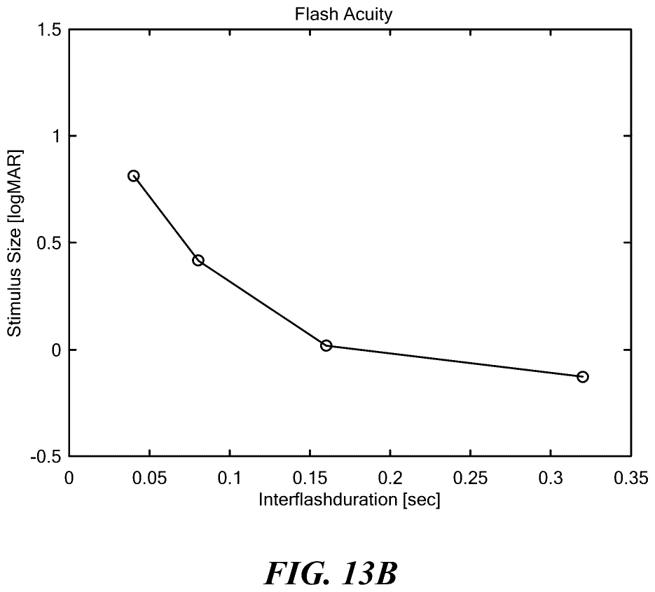

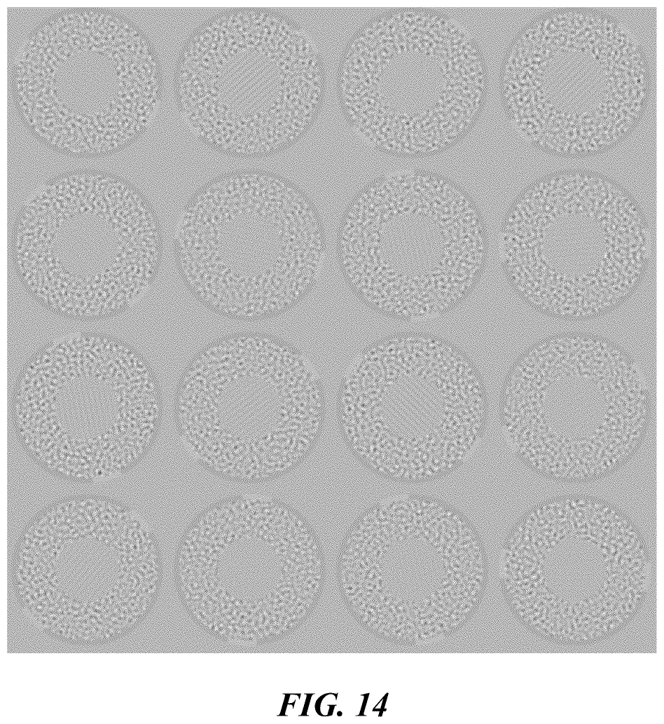

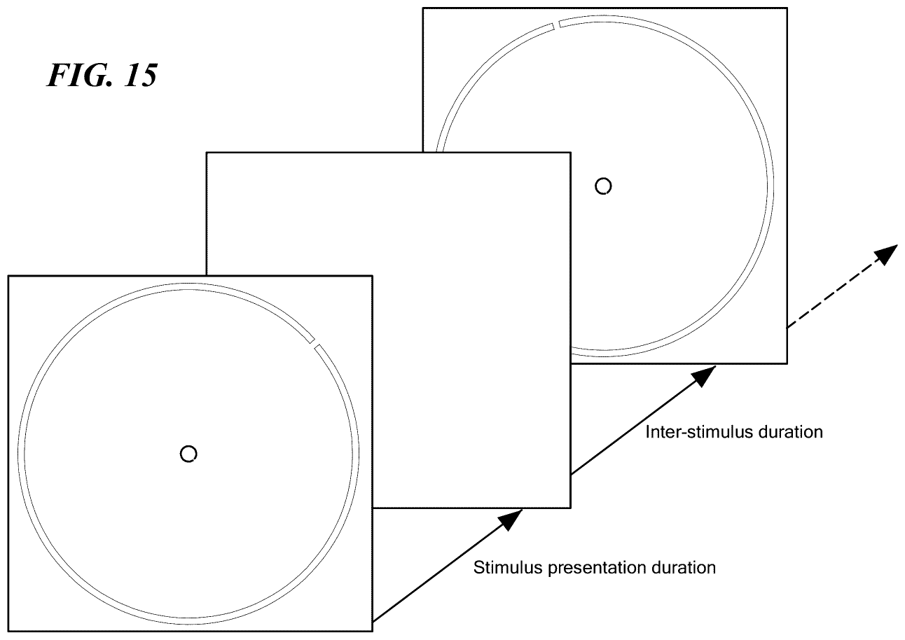

In addition to axial optical aberrations, namely myopic and hyperopic defocus, refractive error may generate an orientation-specific aberration known as astigmatism. The perceptual consequence of astigmatism can be tested with a pattern as illustrated in FIG. 5A. Astigmatism influences the focusing power of the optics of the affected eye at different orientations (see illustrations of simulated distortions as perceived by an observer with astigmatism in FIGS. 5B and 5C) rather than perceiving a uniform circular pattern, one direction is more pronounced, indicating the presence of astigmatism and its orientation). Astigmatism can be corrected with sphero-cylindrical lenses of the correct power and orientation, the present technology Visual Acuity can estimate astigmatism from a post-hoc comparison of sensitivity to the present technology's C targets at different orientations. In case the refraction is unknown, the program can first screen for astigmatism by asking users to adjust the aspect ratio (height:width) and orientation of the window over a radial pattern to create a window that appears circular.

WO 2022/051710 A1 (PCT application PCT/US2021/049250) is hereby incorporated by reference.

### REFERENCES

1. Thulasiraj R D, Aravind S, Pradhan K. Spectacles for the Millions Addressing a priority of “VISION 2020—The Right to Sight” Community Ophthalmol. 2003; 3:19-21.
2. Pan, C.-W., Dirani, M., Cheng, C.-Y., Wong, T.-Y. & Saw, S.-M. The Age-Specific Prevalence of Myopia in Asia: A Meta-analysis. Optom. Vis. Sci. 92, 258-266 (2015).
3. disruptionmag.com/2016/05/17/dave-gilboa-warby-parker/4. He, J., Skerswetat, J., Bex, P. J. 2023. Novel color vision assessment tools: AIM Color Detection and Discrimination. The Association of Research in Vision and Ophthalmology. Abstract accepted
5. Skerswetat, J., Idman-Rait, C., Sun. K., Bex P. J., Ross, N. 2023. Assessment of eleven visual functions in people with and without albinism. The Association of Research in Vision and Ophthalmology. Abstract accepted
6. Ahmed, Z., Skerswetat, J., Bex, P. J., Wiecek, E. K. 2023. Assessment of Visual Acuity in Children With and Without Amblyopia Using the Novel Method AIM (Angular Indication Measurement) Acuity. The Association of Research in Vision and Ophthalmology. Abstract accepted
7. Neupane, S., Skerswetat, J., Bex, P. J. 2023. Validation of Angular Indication Measurement (AIM) Stereoacuity. Vision Science Society Conference. Abstract accepted
8. He, J., Shah, J. B., Skerswetat, J., Bex, P. J. 2023. Refractive Error measured with AIM (Angular Indication Measurement) Visual Acuity. Vision Science Society Conference. Abstract accepted
9. Skerswetat, J. & Bex, P. J . . . 2022. AIM+ (Angular Indication Measurement Plus) enables rapid and self-administered assessment of visual perception dependency across multiple stimulus dimensions. Presented at the European Conference for Visual Perception
10. Skerswetat, J., Boruta, A., Bex, P. J. 2022. Disability Glare Quantified Rapidly with AIM (Angular Indication Measurement) Glare Acuity. Presented at Association for Research in Vision and Ophthalmology.
11. Freeman, M., Skerswetat, J., Bex, P. J. 2022. Measuring the Effect of Negative and Positive Defocus on Visual Acuity Using AIM (Angular Indication Measurement) Acuity. Presented at Virtual Vision Sciences Society conference

1. A method for self-administered testing of one or more visual functions of a subject, comprising the steps of:
(a) providing a device having a graphical display and a user input;
(b) displaying a series of cells on the display; wherein the cells each comprise a visual stimulus having a variable stimulus feature; wherein each cell comprises the visual stimulus disposed within a perimeter feature; wherein the visual stimulus comprises an angular indicator that varies from cell to cell in angular position with respect to the perimeter feature, each different angular position associated with a variation of the variable stimulus feature correlated with a visual function feature;
(c) receiving subject responses through the user input, the responses indicating the subject's selection, for each cell, of a position on the perimeter feature of the cell corresponding to the angular indicator of the stimulus of the cell; and
(d) analyzing the subject responses to obtain a measure of said visual function for the subject.

2. The method of claim 1, wherein the variable stimulus feature varies from cell to cell with respect to one or more of luminance, contrast, color, perceived depth, motion, flicker, spatial form, object recognition, center-surrounded stimulus, disability surrounded stimulus, object shape, object form, object size, stimulus feature position, stimulus feature angle, perceived interocular suppression, spatial resolution, spatial frequency, noise-defined depth, and sparse-pattern depth, presented either in the central or peripheral visual field.

3. The method of claim 1, wherein the variable stimulus feature varies within the series of cells over a range from difficult-to-detect to easy-to-detect for the subject.

4. The method of claim 1, wherein the series of cells is presented as one or more grids, each grid comprising two or more cells and sharing a common visual stimulus and variable stimulus feature, wherein the variable stimulus feature covers a range of values within each grid.

5. The method of claim 1, wherein the visual stimulus is a Landolt C, and the variable stimulus feature is selected from the group consisting of size, color, contrast to background, fill pattern and/or color, thickness, ratio of gap to thickness, and combinations thereof.

6. The method of claim 1, wherein the visual stimulus is an array or grating of structures comprising dots, bars, lines, or structures having other shapes, and wherein the variable stimulus feature is size, color, contrast to background, contrast to other structures in the array or grating, alignment of structures within the array or grating or a combination thereof.

7. The method of claim 4, wherein artificial intelligence is used to adapt the visual stimulus, variable stimulus feature, or its range of variation to one or more previous responses of the subject, or information about the subject.

8. The method of claim 1, wherein a set of two or more grids are displayed sequentially, and each grid differs from others in the set by a type of visual stimulus or range of variation of the variable stimulus features.

9. The method of claim 8, wherein a blank screen is displayed for a variable time interval between screens containing grids.

10. The method of claim 9, wherein the blank screen is white and wherein the variable time interval is varied in the millisecond time domain, for intervals less than one second, and wherein the subject's flash adaptation is measured.

11. The method of claim 1, wherein the subject wears corrective lenses, anaglyph glasses, polarized glasses, a virtual reality headset, or uses a mirror system, two monitors, or any other dichotic-enabling system during testing.

12. The method of claim 1, wherein the method provides a measure of a visual function selected from the group consisting of visual acuity, visual acuity as a function of refractive errors, contrast sensitivity, glare sensitivity, motion perception, pattern perception, color detection, color discrimination, interocular suppression, monocular center, surround suppression, depth perception, form perception, supra-threshold contrast response, supra-threshold color response, equivalent noise thresholds

13. The method of claim 1, further comprising providing the subject with a plurality of corrective lenses having different refractive error correction and/or astigmatism correction, wherein the method is performed with the subject using one or more of the corrective lenses.

14. The method of claim 13, wherein the opthalmic lenses or frames comprising the lenses are coded for recognition by the subject and/or by the device having a graphical display.

15. The method of claim 1, wherein the method is performed by an unassisted subject using a personal computer, laptop computer, tablet computer, or mobile phone in a non-clinical setting.

16. The method of claim 1, wherein results of the method are reported to a testing organization, ophthalmic optician, optometrist, ophthalmologist, psychologist, psychiatrist, neurologists, or medical doctor.

17. The method of claim 1, wherein a prescription for corrective glasses, contact lenses, or refractive surgery for the subject is produced.

18. The method of claim 1, wherein the test is optimized and personalized for the subject by performing two or more trials of the set of grids, wherein the range of the variable stimulus feature on the first trial are based on data from previous observers or on physical stimulus limits of the display, and wherein the range of the variable stimulus feature on subsequent trials is based on results from all previous grids for the subject.

19. The method of claim 1, wherein two are more variable stimulus features are varied simultaneously over the cells of a grid.

20. The method of claim 1, wherein the method is repeated after one or more time intervals.

21. The method of claim 1, wherein said analyzing comprises use of an angular error function.

22. The method of claim 1, wherein said analyzing comprises measuring the subject's orientation error bias as a function of target orientation.

23. The method of claim 1, wherein a plurality of visual functions are tested, and wherein said analyzing comprises performing cluster analysis.

24. The method of claim 1, wherein said analyzing comprises using a 2D or 3D mathematical function that describes the visual function to extract or predict an aspect of the subject's visual function from data supplied by the subject.

25. The method of claim 24, wherein the 2D or 3D mathematical function describes contrast, spatial frequency, hue, or temporal stimulus change periods.

26. The method of claim 1, wherein the method is performed to aid in diagnosis of the presence, absence, or progression of one or more conditions selected from the group consisting of refractive error, keratoconus, amblyopia, age-related macular degeneration, glaucoma, diabetic retinopathy, color vision deficit, cataract, stroke, traumatic brain injury, brain lesion area V4, brain lesion area MT, brain lesion area FFA, Alzheimer's disease, Parkinson's disease, prosopagnosia, object agnosia, autism spectrum disorder, attention deficit disorder, neurometric response, psychotic disorders, post-traumatic stress disorder, obsessive compulsive disorder, Big 5 personality traits, albinism, prescription drug side effects, multiple sclerosis, visual snow syndrome, and retinitis pigmentosa.

27. A device for performing the method of claim 1, the device comprising a graphic display, a user input, a processor, a memory, optionally wherein the processor and/or memory comprise instructions for performing said method.
